(19) United States
(12) Patent Application Publication
(10) Pub. No.: US 2015/0133718 A1
(43) Pub. Date: May 14, 2015

(54) HINGED TRANSCRANIAL MAGNETIC STIMULATION ARRAY FOR NOVEL COIL ALIGNMENT

(71) Applicant: CERVEL NEUROTECH, INC., Foster City, CA (US)

(72) Inventors: M. Bret Schneider, Portola Valley, CA (US); John W. Sadler, Belmont, CA (US); Brian Becky, Portola Valley, CA (US); Ai-Ting Stephanie Yang, San Francisco, CA (US)

(21) Appl. No.: 14/398,042

(22) PCT Filed: May 3, 2013
(86) PCT No.: PCT/US2013/039537

§ 371 Date: Oct. 30, 2014

Related U.S. Application Data
(60) Provisional application No. 61/642,290, filed on May 3, 2012.

(51) Int. Cl. A61N 2/02; A61N 2/00

(52) U.S. Cl. CPC A61N 2/02; A61N 2/006
USPC 600/14

(57) ABSTRACT

Adjustable devices and methods of adjusting and customizing Transcranial Magnetic Stimulation (TMS) electromagnets to produce an elongated path of induced electrical current along a user-defined trajectory in patients having a variety of different head shapes and curvatures. Existing TMS electromagnets (“coils”) allow only limited adjustment of the current delivery surfaces. The present invention provides means for adjusting the sub-coil loops within a double coil structure. The present design may be powered with the use of a single TMS pulse generator unit.

### CROSS REFERENCE TO RELATED APPLICATIONS

This patent application claims priority to provisional patent application No. 61/642,290, filed on May 3, 2012. This provisional patent application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

Described herein are Transcranial Magnetic Stimulation (TMS) methods, devices and systems. In particular, TMS coils that are adjustable to a patient's head are described. Systems may include TMS electromagnets with a pair of coils that are adjustable so that the angle between the coils can be changed and held in selected positions.

### BACKGROUND OF THE INVENTION

Typical transcranial magnetic stimulation (TMS) electromagnets may include multiple fixed coils forming the body of the magnet. Such TMS coil designs have usually been designed for either high focality or deep penetration, and are not specifically configured either to target a specific brain region of to target this brain region even when applied to different users.

For example, the TMS coils described in the patent applications listed below are directed to deep brain TMS methods, including electromagnets. These TMS electromagnets include different designs: U.S. patent application Ser. No. 12/669,882, titled “DEVICE AND METHOD FOR TREATING HYPERTENSION VIA NON-INVASIVE NEUROMODULATION,” and filed on Jun. 2, 2010; U.S. patent application Ser. No. 12/671,260, titled “GANTRY AND SWITCHES FOR POSITION-BASED TRIGGERING OF TMS PULSES IN MOVING COILS”, and filed on Jun. 17, 2010; U.S. patent application Ser. No. 12/670,938, titled “FIRING PATTERNS FOR DEEP BRAIN TRANSCRANIAL MAGNETIC STIMULATION,” and filed on Jun. 17, 2010; U.S. patent application Ser. No. 12/677,220, titled “FOCUSED MAGNETIC FIELDS,” and filed on Sep. 16, 2010; U.S. patent application Ser. No. 12/679,960, titled “DISPLAY OF MODELED MAGNETIC FIELDS,” and filed on Sep. 15, 2010; U.S. patent application Ser. No. 12/680,749, titled “INTRA-SESSION CONTROL OF TRANSCRANIAL MAGNETIC STIMULATION,” and filed on Jul. 14, 2010; U.S. patent application Ser. No. 12/680,912, titled “TRANSCRANIAL MAGNETIC STIMULATION WITH PROTECTION OF MAGNET-ADJACENT STRUCTURES,” and filed on Jul. 14, 2010; U.S. patent application Ser. No. 12/324,227, titled “TRANSCRANIAL MAGNETIC STIMULATION OF DEEP BRAIN TARGETS,” and filed on Nov. 26, 2008; U.S. patent application Ser. No. 12/990,235, titled “TRANSCRANIAL MAGNETIC STIMULATION BY ENHANCED MAGNETIC FIELD PERTURBATIONS,” and filed on Nov. 30, 2010; U.S. patent application Ser. No. 12/185,544, titled “MONOPHASIC MULTI-COIL ARRAYS FOR TRANSCRANIAL MAGNETIC STIMULATION,” and filed on Aug. 4, 2008; U.S. patent application Ser. No. 12/701,395, titled “CONTROL AND COORDINATION OF TRANSCRANIAL MAGNETIC STIMULATION ELECTROMAGNETS FOR MODULATION OF DEEP BRAIN TARGETS,” and filed on Feb. 5, 2010; U.S. patent application Ser. No. 13/141,100, titled “SHAPED COILS FOR TRANSCRANIAL MAGNETIC STIMULATION,” and filed on Aug. 1, 2011; U.S. patent application Ser. No. 12/838,299, titled “TRANSCRANIAL MAGNETIC STIMULATION FIELD SHAPING,” and filed on Jul. 16, 2010; U.S. patent application Ser. No. 12/912,650, titled “SUB-MOTOR-THRESHOLD STIMULATION OF DEEP BRAIN TARGETS USING TRANSCRANIAL MAGNETIC STIMULATION,” and filed on Oct. 26, 2010; and U.S. patent application Ser. No. 13/169,967, titled “ENHANCED SPATIAL SUMMATION FOR DEEP-BRAIN TRANSCRANIAL MAGNETIC STIMULATION,” and filed on Jun. 27, 2011.

The inventors herein suggest, based on ongoing experimental data, that there may be clinical efficacy in deliberately steering the direction of induced current to a particular brain region, and in particular, directing induced current across a distance that may traverse two or more structures involved in a targeted brain circuit.

However, the fixed-coil TMS electromagnets currently available are not configured to produce an elongated path of induced electrical current along a pre-defined trajectory while adapting to differences in the curvature of each patient's head. For example, even the more physically flexible, large-coil designs currently available stimulate too much brain tissue lateral to the targeted strip, and thereby raise safety concerns. Thus, there is a need for TMS electromagnets (“coils”) that stimulate along an extended linear trajectory while conforming to the unique curvature of each patient's head.

Described herein are TMS electromagnets and methods of using them to treat patients that may address the concerns raised above.

### SUMMARY OF THE INVENTION

In general, described herein are TMS electromagnets that are configured to target specifically and exclusively a predefined target brain region, even when applied to patients having different head shapes. Thus, the TMS electromagnets may be configured to adjust to different patient head geometries, while targeting the same specific brain region (and not substantially targeting other brain regions. Also described herein are methods of using such TMS electromagnetic devices.

For example, described herein are TMS electromagnet device(s) that are configured to provide transcranial magnetic stimulation to a specified area of the brain, wherein the specific area of the brain is a longitudinal strip extending from the left dorsolateral prefrontal cortex to the medial frontal cortex overlying the dorsal anterior cingulate gyrus; these TMS electromagnets may target this same region a variety of head sizes and shapes using the same coil apparatus. This TMS electromagnet (coil apparatus) may therefore be adjustable to different head sizes while maintaining the same brain target region(s).

In some variations a Transcranial Magnetic Stimulation (TMS) electromagnet device may include: an adjustable head frame that is configured to be worn on the patient's head and holds at least one TMS electromagnet that is (or can be) oriented to stimulate a predetermined target brain region without substantially stimulating more laterally positioned regions; and a TMS electromagnet that is adjustably connected to the adjustable head frame, wherein the TMS electromagnet comprises a first TMS coil and a second TMS coil that are adjustably and electrically connected to each other so that the angle between the first and second TMS coils may be adjusted.

In general, a TMS electromagnet device as described herein may also include a second (or third, fourth, fifth, etc.) TMS electromagnet that is adjustably connected to the adjustable head frame, wherein the second TMS electromagnet comprises a third TMS coil and a fourth TMS coil that are adjustably and electrically connected to each other so that the angle between the third and fourth TMS coils may be adjusted. Each of the pair of TMS coils may include multiple windings of a material used to form the TMS electromagnet, and the two coils may be connected so that the windings of each are electrically connected (and continuous), so that the TMS electromagnet functions as a unit. As shown and descried in greater detail herein, these TMS electromagnets (which may be referred, for convenience, as “hinged TMS electromagnets” or “angle adjustable TMS electromagnets”) typically include a region between each coil that permits the two coils to be adjusted relative to each other. For example, the first coil may be bent, twisted, rotated, angled, etc. The adjustment may be along a line (e.g., hinged motion) or a point (e.g., pivoting), so that the planes of each coil move relative to each other.

For example, the TMS electromagnet may be configured so that the angle between the plane of the first TMS coil and the plane of the second TMS coil may be adjusted from a hinge region between the first TMS coil and the second TMS coil. In some variations, the TMS electromagnet is configured so that the angle between the plane of the first TMS coil and the plane of the second TMS coil may be adjusted from a pivot point between the first TMS coil and the second TMS coil.

In some variations, the TMS device includes a lock, holder, or other securement that is configured to hold the angle between the first TMS coil and the second TMS coil of the TMS electromagnet once it has been adjusted. The lock/holder may be released and re-secured, or in some variations it may be permanent. The lock may be secured by screwing, or otherwise engaging a member, pin, clasp, etc.

The adjustable head frame devices may be configured to target any appropriate brain region. For example, the devices may be configured to aim TMS at a predetermined brain region such as the left dorsolateral prefrontal cortex to medial frontal cortex overlapping the dorsal anterior cingulate gyrus. The adjustable head frame may include an adjustable headband. The

TMS electromagnets may be held to the adjustable head frame by one or more holders that retain the TMS electromagnet(s) on the head frame, but allows it to be adjusted so that the shape of the TMS electromagnets may be conformed to the subject's head. The holder(s) may be configured so that they generally aim the TMS electromagnets to a predetermined target, but allow just enough movement of the TMS electromagnets so that they can conform to the head.

Thus also generally described herein are adjustable Transcranial Magnetic Stimulation (TMS) electromagnets (adjustable TMS electromagnet devices), that may include: a first TMS coil comprising multiple coil windings; a second TMS coil comprising multiple coil windings; an adjustable connecting region between the first TMS coil and the second TMS coil, wherein the adjustable connecting region is configured so that the angle between the first and second TMS coils may be adjusted; and a lock configured to hold the adjustable connecting region in a predetermined position, wherein the first TMS coil and the second TMS coil are electrically connected through the adjustable connecting region so that current flows between the first TMS coil and the second TMS coil. Although TMS electromagnets having two coils are described and shown herein, it should be understood that these devices may include three, four, or more coils; these coils may be connected at a single connecting region or multiple adjustable connecting regions may be included.

As mentioned, any of these devices may include a hold or lock configured to lock the angle between the coils (e.g., the first TMS coil and the second TMS coil) of the TMS electromagnet once it has been adjusted.

In some variations, the adjustable connecting region may comprise a hinge region configured so that the angle between a plane of the first TMS coil and a plane of the second TMS coil may be adjusted from the hinge region. In some variations, the adjustable connecting region comprises a pivot region (which may be a pivot point) configured, e.g., so that the angle between the plane of the first TMS coil and the plane of the second TMS coil may be adjusted from the pivot region.

Also described herein are coil devices for Transcranial Magnetic Stimulation (TMS), the device comprising: a curved undersurface configured to approximate the curvature of a human skull; an outwardly extending portion for either drawing or returning current to/from the curved undersurface; an inwardly extending region for either drawing or returning current to/from curved undersurface. In some variations the inwardly and outwardly portions are joined in electrical contact. In some variations, the curved undersurface comprises a flexible conductive material that conforms to the shape of patient's skull when in the specified position.

### BRIEF DESCRIPTION OF THE DRAWINGS

FIG. 1 illustrates a set of two coils of a TMS electromagnet with a hinge apparatus retaining the two in proximity, and with underlying hemi-pads keeping the coils at an appropriate tilt, such that the main direction of primary electrical current within both coils is aligned diagonally from the left prefrontal cortex to the midline over the dorsal cingulate.

FIG. 2 illustrates multi-axis hinge mechanisms that permit a pair of coils to contact the scalp of varying head shapes and sizes, only along those portions of the coil circumference that induce current in the desired direction.

FIG. 3 details the hemi-padding arrangement that places one portion of each coil surface closer to the scalp than the other portions of each coil surface.

FIG. 4A illustrates an arrangement in which each of the two coils are physically bent, thereby placing one portion of each coil surface closer to the brain than the other portions of each coil surface, without the requirements for pads.

FIG. 4B illustrates an exemplary electrical wiring approach for the two coils, whereby they are powered in parallel by electrical pulses discharged between the positive and negative terminals of a single standard TMS pulse generator.

FIG. 4C illustrates the two tiltable hemi-padded coils powered by a single TMS pulse generator, with an arrow indicating the principal direction of the primary electrical current within the coils on the portions of the coil which are placed closest to the brain.

FIG. 5 illustrates a hinged coil pair attached to a suspension apparatus, in which multiple axes of positioning movement are enabled.

FIG. 6 illustrates a ball and socket mechanism for allowing relative positioning of the two circular coil elements.

FIG. 7 illustrates a cross wire “x-wire” mechanism for allowing relative positioning of the two circular coil elements.

FIG. 8 illustrates a trolley-in-rail mechanism for allowing relative positioning of the two circular coil elements.

FIG. 9 illustrates a flexible “sock”-like fitting that joins the two coils, allowing relative positioning.

FIG. 10A represents a “D-shaped” coil design with a curved undersurface and specified placement in which a concentrically wound single coil powered by a single TMS pulse source. Space between coil windings is exaggerated for illustrative purposes.

FIG. 10B specifies the location and direction of primary electrical current within the coil at the scalp-contacting curved undersurface of the coil, as related to head positions defined in accordance with the EEG 10-20 convention. Induced current within the brain moves in a direction opposite that of the primary electrical current in the coil.

FIG. 10C illustrates a lateral projection of a patient's head with the D-coil in place, with one end over the left dorsolateral prefrontal cortex, turning posteriorly at a diagonal, and the other end over midline above dorsal anterior cingulate.

FIG. 10D illustrates an anterior-posterior projection of a patient's head with the D-coil in place, with one end over the left dorsolateral prefrontal cortex, turning posteriorly at a diagonal, and the other end over midline above dorsal anterior cingulate.

### DETAILED DESCRIPTION OF THE INVENTION

In general, the devices described herein include one or more TMS electromagnets that are configured to be worn on a patients head to induce current in a specific target region of the patient's brain (e.g., the left dorsolateral prefrontal cortex overlaying the dorsal anterior cingulate gyrus). The devices maybe configured to stimulate this region specifically (and without substantially stimulating non-target regions) in a variety of head sizes and shapes using the same device.

In some variations this device includes a head mount holding the TMS electromagnet, which may be configured as a hat, helmet, headband, or the like. The position or orientation of the TMS electromagnets (which may include two or more TMS electromagnets or coils) may be fixed, while various subcomponents of the TMS electromagnets may be adjustable.

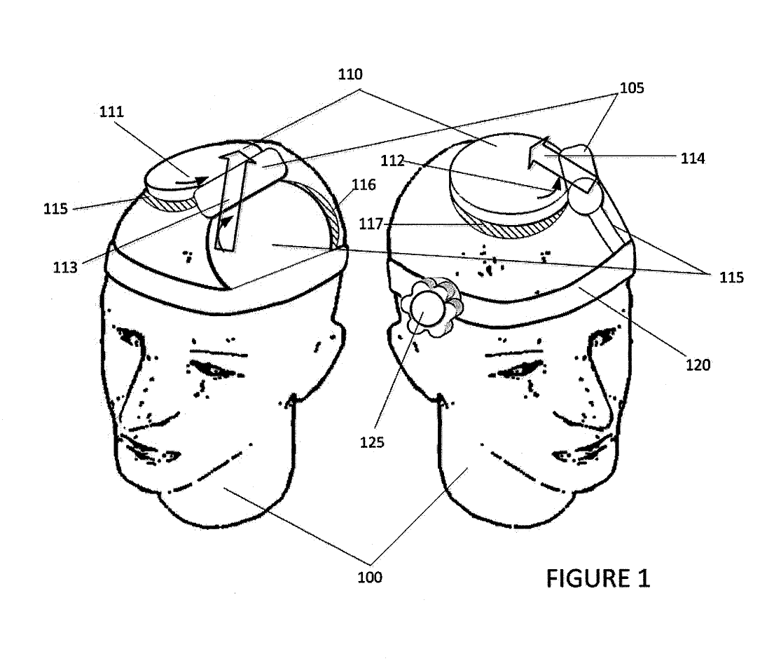

For example, FIG. 1 illustrates a set of two coils with a hinge apparatus retaining the two in proximity, and with underlying hemi-pads keeping the coils at an appropriate tilt, such that the main direction of primary electrical current within both coils is aligned diagonally from the left prefrontal cortex to the midline over the dorsal cingulate. In this example, the patient's head 100 is shown, the patient is wearing a headband 120, to which a top coil portion 110 and a left coil portion 115 are attached, separated by a coil hinge 105. The device or system shown in this embodiment is specifically arranged to be worn so that the direction of primary current within the coils at the scalp-contacting surface of the coil (113 and 114) is oriented to specifically and narrowly stimulate the left dorsolateral prefrontal cortex overlaying the dorsal anterior cingulate gyrus. The figure also illustrates displacement hemi-pads 115, 116, and 117 on the headband 120; the headband 120 also includes one or more adjustable controls (ratchet 125) that may be used to tighten/loosen the headband on the head. In this example, the direction of primary current in coil section 111 and 112 is indicated by the curving arrows.

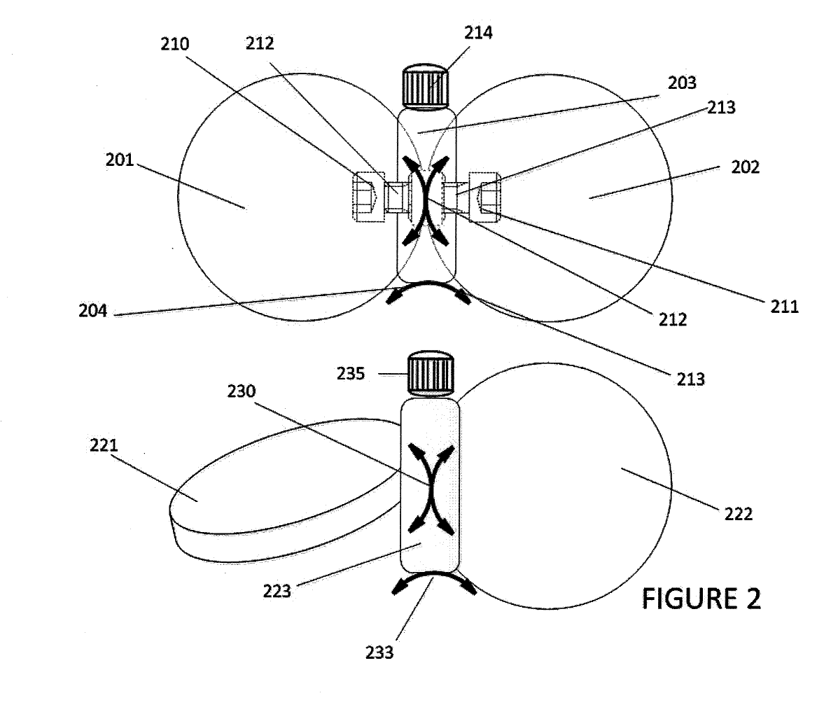

Another device example is shown in FIG. 2, which illustrates a multi-axis hinge mechanisms that permit a pair of coils to contact the scalp of varying head shapes and sizes, only along those portions of the coil circumference that induce current in the desired direction. In this example the TMS electromagnets shown may be used with any appropriate holder or head mount, such as the headband shown in FIG. 1. FIG. 2 shows: a first coil 201 and 221, a second coil 202 and 222, a coil roll hinge 203, a pitch hinge 211 and 210, and a hinge ratchet lock 214 and 235. FIG. 2 also illustrates the direction of pitch 211 and 230, direction of roll 204 and 233, and shows pivot portions 212 and 213. The angle and orientation of the TMS electromagnets (coils) may be adjusted as indicated; the angle between each of the two coil regions (e.g., 201 and 222) may be separately adjusted.

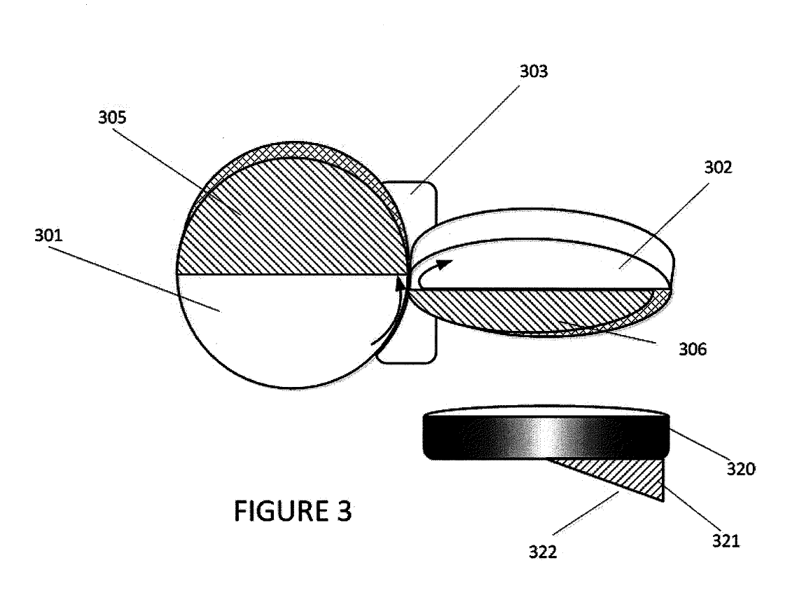

FIGS. 3 through 9 illustrate other variations of similarly adjustable TMS electromagnets.

For example, FIG. 3 details a hemi-padding arrangement that places one portion of each coil surface closer to the scalp than the other portions of each coil surface. In this example, the Figure shows: a first coil 301, a second coil 302, a first hemi-pad 305, a second hemi-pad 306, and a hinge 303. The lower right portion of the figure shows a lateral view of coil 320, including a lateral view of hemi-pad 321, and a patient-contacting surface 322.

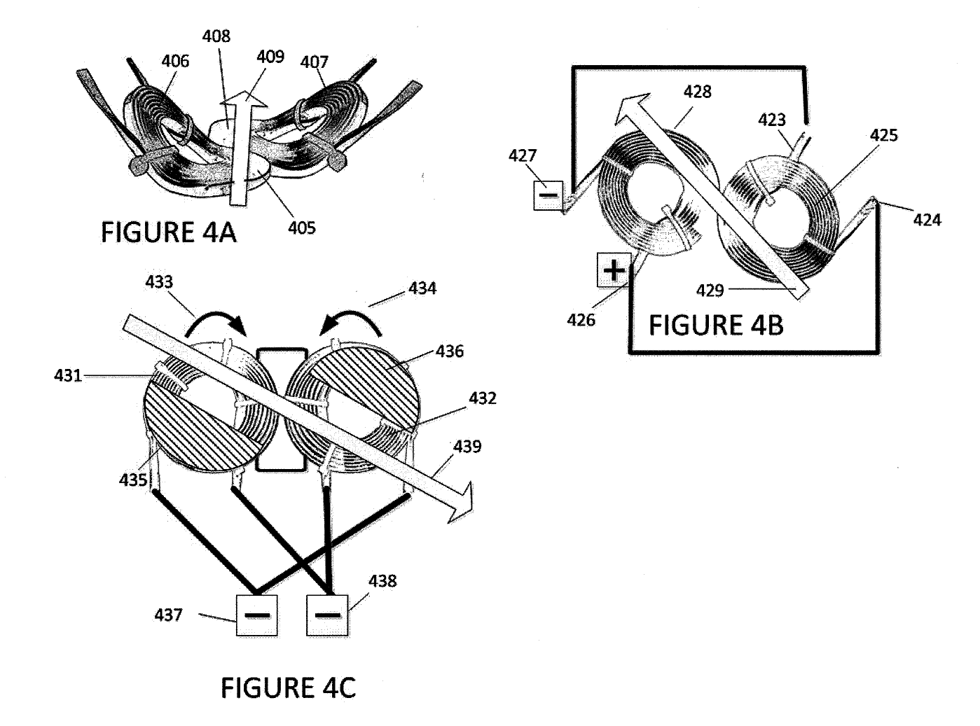

FIG. 4A illustrates an arrangement in which each of the two coils of the TMS electromagnet are physically bent, thereby placing one portion of each coil surface closer to the brain than the other portions of each coil surface, without the requirements for pads. The first bent coil section 406, second bent coil section 407, first patient-contacting surface of coil 405, and second patient-contacting surface of coil 408 are shown, along with the direction of primary current in coils at contacting surface 409.

FIG. 4B illustrates an exemplary electrical wiring approach for the two coils, whereby they are powered in parallel by electrical pulses discharged between the positive and negative terminals of a single standard TMS pulse generator. In this example, negative pulse generator terminal 427, positive pulse generator terminal 426 contact the first coil 428 and second coil 425 at positive lead of first coil 424 and negative lead of first coil 423. The direction of primary current in coils 429 is also illustrated by the large arrow.

FIG. 4C illustrates the two tiltable hemi-padded coils powered by a single TMS pulse generator, with an arrow indicating the principal direction of the primary electrical current within the coils on the portions of the coil that are placed closest to the brain. In FIG. 4C, the negative pulse generator terminal 438, positive pulse generator terminal 437, first coil 431, second coil 432, first hemi-pad 435, and second hemi-pad 436 are all illustrated. The primary direction of current in first coil 433 as well as the direction of primary current in second coil 434 and direction of primary current of both coils at patient contacting surface 439 are shown.

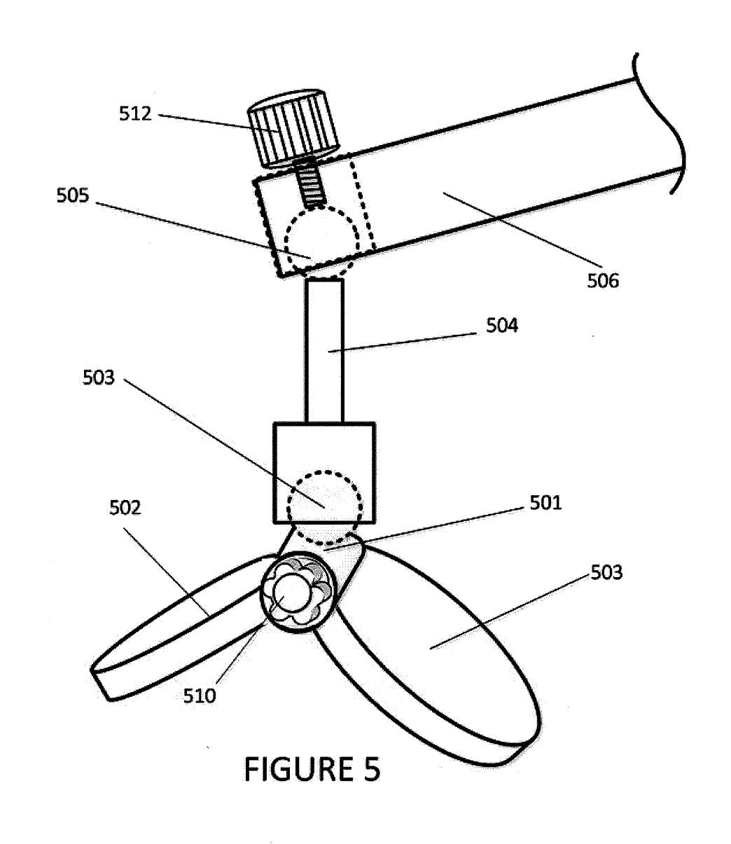

FIG. 5 illustrates a hinged coil pair attached to a suspension apparatus, in which multiple axes of positioning movement are enabled. In this example, the following elements are shown in their relative relationships: ratcheted hinge 501, ratchet lock knob 510, first coil 502, second coil 503, ball in socket 503, extension shaft 504, second ball in socket 505, fixation knob 512, and cantilevered arm 506.

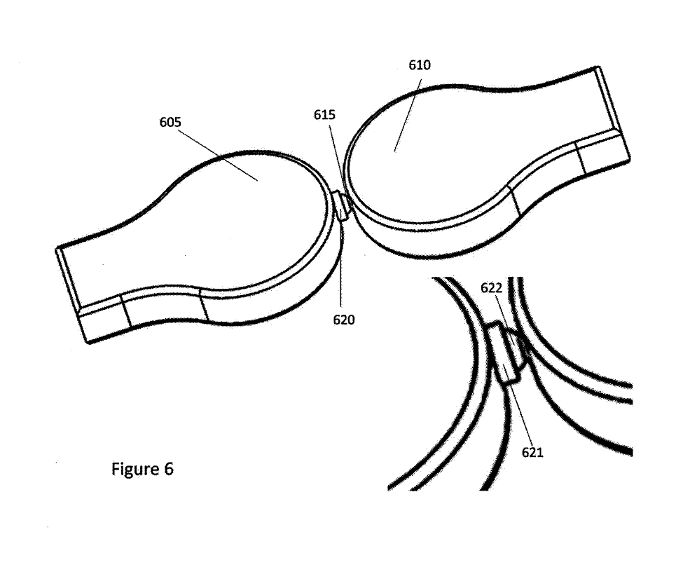

In FIGS. 6-9, various mechanisms illustrate ways to allow relative positioning of the two circular coil elements. Space between first and second coil shown is exaggerated for concept illustration purposes. Hinge mechanisms may also be of larger sturdier dimension, but are shown here in small scale for better visibility in the context of the whole diagram. It is intended that these illustrative mechanisms be combined with sturdy mechanical fixation means that prevents unintended loss of their affixed position with patient movement and coil pulsing. Such fixation means may include ratcheting, friction-based fixation, motorized stepping, and pin and hole locks.

For example, FIG. 6 illustrates a ball and socket mechanism for allowing relative positioning of the two circular coil elements. In this example, the first coil enclosure 605, second coil enclosure 610, ball 615, and socket 620. The inset region in the lower right shows a close up of ball 622 and a close up of socket 621.

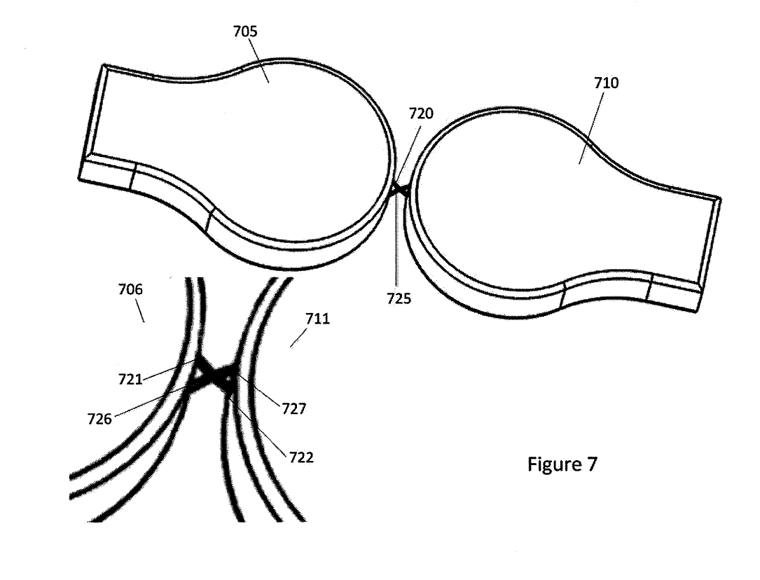

FIG. 7 illustrates a cross wire “x-wire” mechanism for allowing relative positioning of the two circular coil elements, including a first coil enclosure 705, second coil enclosure 710, first cross-wire 720, and second cross-wire 725. The inset region in the lower left corner shows a close up of first coil enclosure 706, a close up of second coil enclosure 711, a close up of first cross-wire 721, a close up of second cross-wire 726, an attachment of second cross-wire to second coil enclosure 727, and an attachment of first-cross wire to second coil enclosure 722.

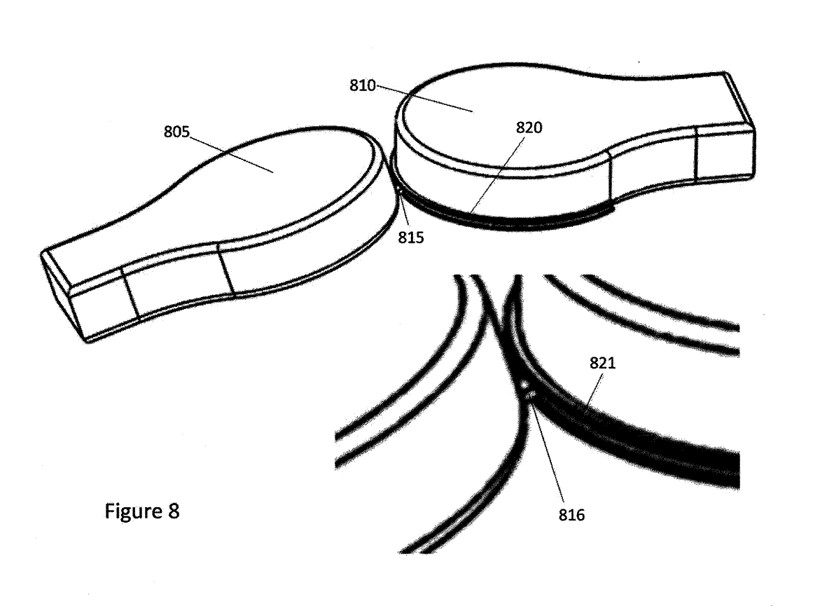

FIG. 8 illustrates a trolley-in-rail mechanism for allowing relative positioning of the two circular coil elements. In this example the TMS electromagnet includes a first coil enclosure 805, a second coil enclosure 810, a trolley 815, a track 820, and in the lower right inset region, a close up of trolley 816, a close up of track 821.

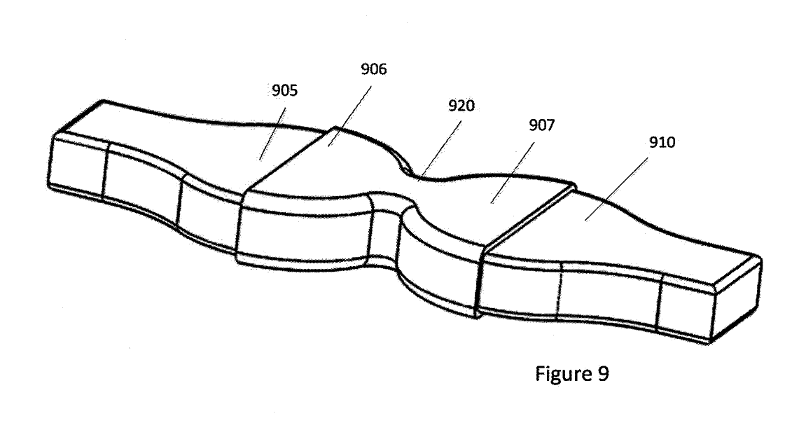

FIG. 9 illustrates a flexible “sock”-like fitting that joins the two coils, allowing relative positioning. In FIG. 9, a first coil exterior 905, second coil exterior 910, first portion of flexible sock 906, and a second portion of flexible sock 907 as well as an intermediate portion of flexible sock 920 are shown.

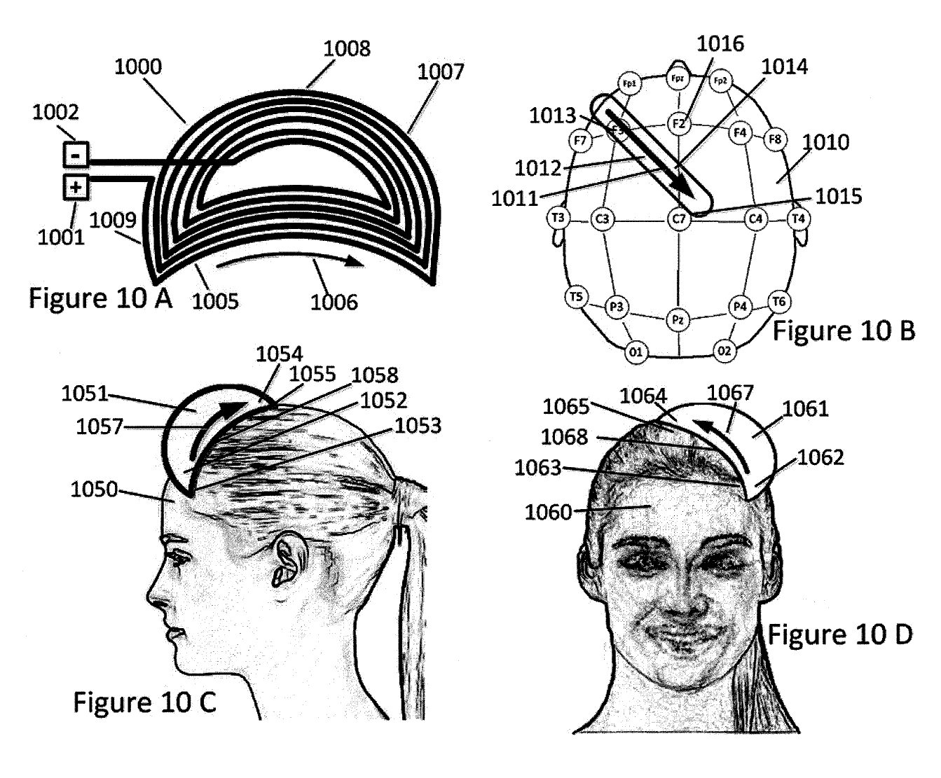

FIG. 10A shows a represents a “D-shaped” coil design with a curved undersurface and specified placement in which a concentrically wound single coil powered by a single TMS pulse source. FIG. 6A shows the general orientation of the insulated conductive members, with exaggerated space between the coil windings for illustrative purposes: in reality these concentric conductive members are intended to be as tightly wound as possible for inductive efficiency. Curved undersurface 1005 may be made of either rigid material such as copper and potting material, or may be made of flexible material such as stranded copper or silver cable. This portion may also be constructed with Litz wire or similar flexible conductive material. (Expand detailing each item numbered in the figure).

FIG. 10A shows a TMS electromagnet coupled or coupleable to a positive terminal of pulse generator 1001, a negative terminal of pulse generator 1002. The TMS electromagnet includes a “D” coil 1000 having a free transverse portion 1008, upwardly extending portion 1007, downwardly extending portion 1009, and a patient contacting surface 1005. FIG. 10A also shows the direction of primary current in patient-contacting surface 1006.

FIG. 10B specifies the location and direction of primary electrical current within the coil at the scalp-contacting curved undersurface of the coil, as related to head positions defined in accordance with the EEG 10-20 convention. Induced current within the brain moves in a direction opposite that of the primary electrical current in the coil. Preferred placement for the coils described herein (including the D-shaped coil of FIG. 10A) is with a posterior slanted diagonal from the left side of the head near F3, to the top of the head anterior to C7 (1015), but posterior to F2 (1016). In this manner, conventional electrical current flows within curved undersurface 1005 from the left side of the head at a posterior slanted diagonal to the top of the head overlying medial frontal cortex, and dorsal anterior cingulate below it.

In FIG. 10B, the schematic of EEG 10-20 layout of a head 1010 includes a point F2 1016, CZ 1015, and indicates the footprint of the “D” coil on patient's head 1012, spanning the point F3 1013. The direction of primary current in “D” coil at patient-contacting surface is indicated by the arrow 1011.

The positive side 1052 of curved undersurface 1005 is placed approximately over the F3 (EEG 10-20 nomenclature), or the left dorsolateral prefrontal cortex, Brodmann's Area 9/46. The negative side 1054 of curved undersurface 1005 is placed over medial frontal cortex, anterior to C7 (1015), but posterior to F2 (1016). In this manner, conventional electrical current flows within the curved undersurface 605 from the left side of the head.

FIG. 10C illustrates a lateral projection of a patient's head with the D-coil in place, with one end over the left dorsolateral prefrontal cortex, turning posteriorly at a diagonal, and the other end over midline above dorsal anterior cingulate. Preferred placement for this coil is with the positive side 1052 of curved undersurface 1005 is placed approximately over the F3 (EEG 10-20 nomenclature), or the left dorsolateral prefrontal cortex, Brodmann's Area 9/46. The negative side 1054 of curved undersurface 1005 is placed over medial frontal cortex, anterior to C7 (615), but posterior to F2 (616). In this manner, conventional electrical current flows within the curved undersurface 1005 from the left side of the head in the desired region. In this example, the patient 1050 is shown wearing the “D” coil 1051. The “D” coil includes an inferior margin of “D” coil 1052, a superior margin of “D” coil 1054. The top of head, anterior 2 CZ 1055, F3 1053 are shown. The direction of primary current in coil at patient-contacting surface 1057 is also illustrated.

FIG. 10D illustrates an anterior-posterior projection of a patient's head with the D-coil in place, with one end over the left dorsolateral prefrontal cortex, turning posteriorly at a diagonal, and the other end over midline above dorsal anterior cingulate. Similar to FIG. 10C, FIG. 10D shows a patient 1060 wearing a “D” coil 1061 and include an inferior margin of “D” coil 1062. Region F3 1063, the superior margin of “D” coil 1064, and the top of head anterior to CZ 1065 are all show. In this example, the direction of primary current in coil at patient-contacting surface 1067 is also shown.

Although the description above is broken into parts and includes specific examples of variations of suture passers, any of the features or elements described in any particular example or section may be incorporated into any of the other embodiments. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A Transcranial Magnetic Stimulation (TMS) electromagnet device, the device comprising:
an adjustable head frame configured to be worn on the patient's head and hold at least one TMS electromagnet oriented to stimulate a predetermined target brain region without substantially stimulating more laterally positioned regions; and
a TMS electromagnet adjustably connected to the adjustable head frame, wherein the TMS electromagnet comprises a first TMS coil and a second TMS coil that are adjustably and electrically connected to each other so that the angle between the first and second TMS coils may be adjusted.

2. The device of claim 1, further comprising a second TMS electromagnet adjustably connected to the adjustable head frame, wherein the second TMS electromagnet comprises a third TMS coil and a fourth TMS coil that are adjustably and electrically connected to each other so that the angle between the third and fourth TMS coils may be adjusted.

3. The device of claim 1, further comprising a lock configured to lock the angle between the first TMS coil and the second TMS coil of the TMS electromagnet once it has been adjusted.

4. The device of claim 1, wherein the TMS electromagnet is configured so that the angle between the plane of the first TMS coil and the plane of the second TMS coil may be adjusted from a hinge region between the first TMS coil and the second TMS coil.

5. The device of claim 1, wherein the TMS electromagnet is configured so that the angle between the plane of the first TMS coil and the plane of the second TMS coil may be adjusted from a pivot point between the first TMS coil and the second TMS coil.

6. The device of claim 1, wherein the predetermined target brain region comprises the left dorsolateral prefrontal cortex to medial frontal cortex overlapping the dorsal anterior cingulate gyrus.

7. The device of claim 1, wherein the adjustable head frame comprises an adjustable headband.

8. An adjustable Transcranial Magnetic Stimulation (TMS) electromagnet device, the device comprising:
a first TMS coil comprising multiple coil windings;
a second TMS coil comprising multiple coil windings;
an adjustable connecting region between the first TMS coil and the second TMS coil, wherein the adjustable connecting region is configured so that the angle between the first and second TMS coils may be adjusted; and
a lock configured to hold the adjustable connecting region in a predetermined position,
wherein the first TMS coil and the second TMS coil are electrically connected through the adjustable connecting region so that current flows between the first TMS coil and the second TMS coil.

9. The device of claim 8, further comprising a lock configured to lock the angle between the first TMS coil and the second TMS coil of the TMS electromagnet once it has been adjusted.

10. The device of claim 8, wherein the adjustable connecting region comprises a hinge region configured so that the angle between a plane of the first TMS coil and a plane of the second TMS coil may be adjusted from the hinge region.

11. The device of claim 8, wherein the adjustable connecting region comprises a pivot region configured so that the angle between the plane of the first TMS coil and the plane of the second TMS coil may be adjusted from the pivot region.

12. A coil device for Transcranial Magnetic Stimulation (TMS), the device comprising:
a curved undersurface configured to approximate the curvature of a human skull;
an outwardly extending portion for either drawing or returning current to/from the curved undersurface;
an inwardly extending region for either drawing or returning current to/from curved undersurface.

13. The device of claim 3, further wherein said inwardly and outwardly portions are joined in electrical contact.

14. The device of claim 3, wherein said curved undersurface comprises a flexible conductive material that conforms to the shape of patient's skull when in the specified position.
